(19) United States
(12) Patent Application Publication
(10) Pub. No.: US 2022/0270747 A1
(43) Pub. Date: Aug. 25, 2022

(54) AUTOMATED CLINICAL DECISION DISAMBIGUATION

(71) Applicant: KONINKLIJKE PHILIPS N.V., EINDHOVEN (NL)

(72) Inventor: CORNELIS CONRADUS ADRIANUS MARIA VAN ZON, EVERETT, MA (US)

(21) Appl. No.: 17/629,047

(22) PCT Filed: Jul. 9, 2020
(86) PCT No.: PCT/EP2020/069382

§ 371 Date: Jan. 21, 2022

Related U.S. Application Data
(60) Provisional application No. 62/877,338, filed on Jul. 23, 2019.

(51) Int. Cl. G16H 40/20

(52) U.S. Cl. CPC G16H 40/20

(57) ABSTRACT

A method for allocating and activating healthcare resources includes receiving an automated clinical decision from an automated clinical decision-making (ACDM) engine, determining that an action based on the automated clinical decision should performed during a time-critical period, identifying a clinician available within the time-critical period, determining a rank of the clinician relative to the automated clinical decision for the patient condition, and selecting the action to be performed by the at least one clinician or based on the automated clinical decision. The selection operation is performed based on the rank within the time-critical period, and all or a portion of the aforementioned operations are automatically performed using at least one machine-implemented algorithm.

### TECHNICAL FIELD

This disclosure relates generally to processing information, and more specifically, but not exclusively, to managing the allocation of healthcare resources.

### BACKGROUND

Attempts are being made to improve the quality of care for patients in hospitals, doctor offices, clinics, nursing homes, and other medical facilities. Many of these attempts involve processing patient data derived from diagnostic equipment and electronic medical records. The processed data may be made accessible to clinical staff, using networking and other communication technologies, to assist them in making care decisions. While these network-based approaches have proven useful in some cases, they essentially serve as passive information repositories and thus are of limited value.

Some systems have been developed that attempt to take a more active role in assisting clinicians in providing patient care. These systems (often referred to as clinical decision support (CDS) systems) collect and organize medical knowledge for use by healthcare professionals. However, while CDS systems can make recommendations to clinicians, they fail to take an active role in developing and managing clinical decisions, ultimately leaving the actual decision-making process for diagnosis, care, and treatment up to medical professionals. Moreover, CDS systems do not take any action in caring for patients when a qualified clinician is not available to administer treatment.

### SUMMARY

A brief summary of various example embodiments is presented below. Some simplifications and omissions may be made in the following summary, which is intended to highlight and introduce some aspects of the various example embodiments, but not to limit the scope of the invention. Detailed descriptions of example embodiments adequate to allow those of ordinary skill in the art to make and use the inventive concepts will follow in later sections.

In accordance with one or more embodiments, a method for allocating healthcare resources includes (a) receiving information from an automated clinical decision-making (ACDM) engine, the information including an automated clinical decision (e.g., to treat a patient condition, order tests, move a patient to a different ward, etc.); (b) determining that an action based on the automated clinical decision is to be performed during a time-critical period; (c) identifying at least one clinician available within the time-critical period; (d) determining a rank of the at least one clinician relative to the automated clinical decision from the ACDM engine for the patient condition; and (e) selecting the action to be performed by the at least one clinician or based on the automated clinical decision, wherein (e) is performed based on the rank within the time-critical period and (b), (c), (d), and (e) are automatically performed using at least one machine-implemented algorithm.

Operations (b), (c), (d), and (e) may be automatically performed using at least one machine-implemented algorithm without human intervention. Operation (c) may include querying at least one database to identify the at least one clinician available within the time-critical period. Operation (c) may include determining availability of the at least one clinician based on information received from a tracking module. Operation (d) may include querying a database to determine qualifications of the at least one clinician based on the action to be taken for the patent condition. Operation (e) may include selecting the action to be performed within the time-critical period based on the automated clinical decision when the automated clinical decision outranks the at least one clinician, or no qualified clinician is available within the time-critical period.

The method may include generating one or more control signals (e.g., digital commands) to automatically perform the action. The one or more control signals may activate a therapeutic device to automatically perform the action. Operation (e) may include selecting the action to be performed by the at least one clinician within the time-critical period when the at least one clinician is available within the time-critical period and outranks the automated clinical decision for the patient condition. The method may include receiving information indicating that a condition has been satisfied; and selecting the action to be performed based on the automated clinical decision. The condition may include one of the at least one clinician did not acknowledge a notification to perform the action, or the at least one clinician did not perform the action within the time-critical period.

In accordance with one or more embodiments, a non-transitory computer-readable medium stores instructions for causing one or more processors to (a) receive information from an automated clinical decision-making (ACDM) engine, the information including an automated clinical decision to treat a patient condition; (b) determine that an action based on the automated clinical decision is to be performed during a time-critical period; (c) identify at least one clinician available within the time-critical period; (d) determine a rank of the at least one clinician relative to the automated clinical decision from the ACDM engine for the patient condition; and (e) select the action to be performed by the at least one clinician or based on the automated clinical decision, wherein (e) is performed based on the rank within the time-critical period and (b), (c), (d), and (e) are automatically performed using at least one machine-implemented algorithm.

Operation (e) may include selecting the action to be performed within the time-critical period based on the automated clinical decision when: the automated clinical decision outranks the at least one clinician, or no qualified clinician is available within the time-critical period. The instructions may cause the one or more processors to generate one or more control signals to automatically perform the action. The one or more control signals may activate a therapeutic device to automatically perform the action. Operation (e) may include selecting the action to be performed by the at least one clinician within the time-critical period when the at least one clinician is available within the time-critical period and outranks the automated clinical decision for the patient condition.

The instructions may cause the one or more processors to receive information indicating that a condition has been satisfied and select the action to be performed based on the automated clinical decision. The condition may include one of the at least one clinician did not acknowledge a notification to perform the action, or the at least one clinician did not perform the action in the time-critical period.

### BRIEF DESCRIPTION OF THE DRAWINGS

The accompanying figures, where like reference numerals refer to identical or functionally similar elements throughout the separate views, together with the detailed description below, are incorporated in and form part of the specification, and serve to further illustrate example embodiments of concepts found in the claims and explain various principles and advantages of those embodiments.

These and other more detailed and specific features are more fully disclosed in the following specification, reference being had to the accompanying drawings, in which:

FIG. 1 illustrates an embodiment of an automated clinical decision-making system which includes a disambiguation engine;

FIG. 2 illustrates an embodiment of a method for allocating healthcare resources using the disambiguation engine;

FIG. 3 illustrates an embodiment of another method for allocating healthcare resources using the disambiguation engine;

FIG. 4 illustrates an example of information used by the using the disambiguation engine;

FIG. 5 illustrates an example of information used by the using the disambiguation engine; and

FIG. 6 illustrates an embodiment of a processing system for the disambiguation engine.

### DETAILED DESCRIPTION

It should be understood that the figures are merely schematic and are not drawn to scale. It should also be understood that the same reference numerals are used throughout the figures to indicate the same or similar parts.

The descriptions and drawings illustrate the principles of various example embodiments. It will thus be appreciated that those skilled in the art will be able to devise various arrangements that, although not explicitly described or shown herein, embody the principles of the invention and are included within its scope. Furthermore, all examples recited herein are principally intended expressly to be for pedagogical purposes to aid the reader in understanding the principles of the invention and the concepts contributed by the inventor(s) to furthering the art and are to be construed as being without limitation to such specifically recited examples and conditions. Additionally, the term, “or,” as used herein, refers to a non-exclusive or (i.e., and/or), unless otherwise indicated (e.g., “or else” or “or in the alternative”). Also, the various example embodiments described herein are not necessarily mutually exclusive, as some example embodiments can be combined with one or more other example embodiments to form new example embodiments. Descriptors such as “first,” “second,” “third,” etc., are not meant to limit the order of elements discussed, are used to distinguish one element from the next, and are generally interchangeable. Values such as maximum or minimum may be predetermined and set to different values based on the application.

Embodiments described herein relate to managing the allocation of healthcare resources and clinical decisions in a way that provides the best care possible to a patient at a given point in time. To achieve this goal, rule sets created by the hospital and/or machine-learning algorithms determine whether a qualified clinician or an automated clinical decision should be used to treat a patient condition. In order to make this determination, various factors may be taken into consideration including clinician availability, clinician qualifications, location of the clinician relative to the patient, clinician rank relative to automated clinical decision for a given patient condition, patient health status, the nature and urgency of the patient condition, the type of procedure, drugs, or other action required to treat the patient condition, and/or the availability of therapeutic equipment needed for treatment.

In some embodiments, a system and method are provided to generate informed and knowledgeable automated clinical decisions for providing care, treatment, or some other action when the status of a clinician is undetermined or the clinician is unavailable. In addition to generating automated clinical decisions, a disambiguation engine may be used to select the automated decision or clinician for administering care, and in some cases control the implementation of the automated clinical decision using, in part, therapeutic equipment without human intervention. The automated clinical decisions may be reviewed after implementation in order to determine their efficacy. The algorithms may then be updated to generate automated decisions in future situations that are more effective given the wide range of patient conditions possible.

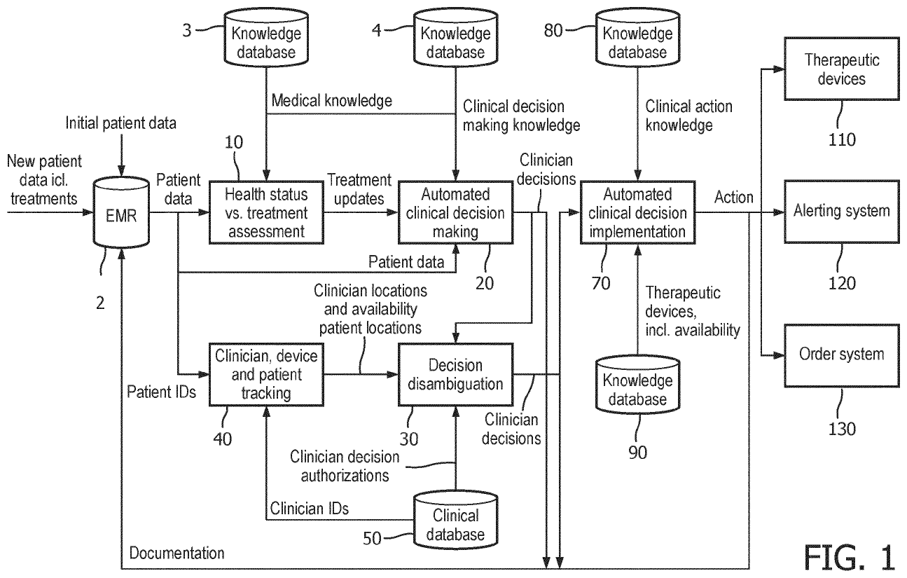

FIG. 1 illustrates an embodiment of an automated clinical decision making (ACDM) system for managing and implementing care decisions for a patient. The care decisions may relate to diagnoses, medical procedures, courses of treatment, tests, administration of drugs, and/or other types of decisions relating to patient care. In order to manage and implement these care decisions, the system may include or be coupled to one or more databases storing information pertinent to or otherwise to be used by a decision engine for making clinical decisions.

The databases include a first database 2, a second database 3, and a third database 4.

The first database 2 may store electronic medical records (EMRs) including past and current medical history, status, and treatment information. This information (which may be referred to as patient data) may be received from a variety of resources within or external to the system. The resources may include, but are not limited to, physician offices, clinics, specialists, hospitals, and/or other medical facilities or personnel who may have been involved in the diagnosis and/or treatment of patients. The information in the EMR database 2 may be received through a network, which, for example, may be a local area network or a wide area network (e.g., Internet). In one embodiment, the network may include a virtual private network, which may or may not be cloud-based.

The second database 3 may store medical knowledge that may be used as a basis for assessing the health of patients and/or the progression and adequacy of their treatments. Examples of the information stored in the medical knowledge database 3 include medical information, biomedical information, genetic information, clinical information, ontologies, terminologies, and vocabularies, e.g., Gene Ontology (GO), Logical Observational Identifiers, Names, and Codes (LOINC), International Classification of Diseases (ICD) 9 or 10, and Systemized Nomenclature of Medicine-Clinical Terms (SNOMED-CT). The medical knowledge database 3 may also store deterministic information, probabilistic information, neural net information, physiological models (e.g., cardio-pulmonary models), and clinical rules (e.g., representing computer-interpretable clinical guidelines or protocols). Other information may be more algorithmic in nature, such as organ status indicators, health status predictors, medical imaging analytics, etc.

The third database 4 stores information that may be used as a basis for making automated clinical decisions. For example, the information stored in database 4 may define what patient data is required to make a clinical decision for a given patient condition. The information may also allow for a determination of the confidence level for a particular automated decision given patient data, assessments, and certain outcomes. The information may also provide an explanation of how the decision was reached.

In one embodiment, the decision-making knowledge stored in database 4 may be codified as a set of rules combined with a Bayesian network, e.g., obtained from a combination of machine learning and expert knowledge. Confidence may be explicitly encoded in the rules. An explanation of how a decision was reached can be obtained, for example, by tracing which rules were triggered and why (e.g., what data caused a rule to be triggered). Techniques for extracting explanations from Bayesian networks may also be used. In one embodiment, the database 4 may also store information indicating treatments, procedures, and/or other actions that may be implemented for various clinical decision that are to be automatically generated by the system.

Referring again to FIG. 1, the ACDM system may include an assessment module 10, an automated clinical decision making (ACDM) engine 20, and a disambiguation engine 30 which perform operations based on the information stored in the aforementioned databases and/or other information stores included in or coupled to the system. The information in the databases may be formalized (e.g., standardized with predetermined fields) in order to allow for access and analysis of the data by the engines and modules of the system. While separate databases 2, 3, and 4 are illustrated to store the different types of information indicated above, a single database may store all of this information in another embodiment, while in yet another embodiment it may be distributed over more than four databases.

The assessment module 10 may determine health status based on patient data stored in the EMR database 2 and medical knowledge stored in database 3. The assessment module may be implemented by an algorithm which, for example, may apply the medical knowledge to the patent data in order to provide an assessment of the current health condition of a patient. In order to make this assessment, the algorithm may indicate what patient data is mandatory and what patient data is optional for purposes of making an assessment. The optional data, if available, may increase the confidence level or accuracy of the assessment and, for example, may vary from condition-to-condition, disease-to-disease, and/or patient-to-patient. The assessment module 10 may also determine the current treatment(s) a patient is undergoing, the phase of treatment the patient is currently in, and/or the progress or improvement of the patient condition realized by that treatment. In some embodiments, given these outcomes, the assessment module 10 may also provide an indication of whether a change of treatment is suggested.

The ACDM engine 20 generates an automated clinical decision for a patient condition. The automated clinical decision may be based on patient data stored in the EMR database 2, medical knowledge stored in database 3, clinical decision making knowledge stored in database 4, and assessment information (including but not limited to any treatment updates) received from the assessment module 10 which applies this knowledge to patient data. In one embodiment, the ACDM engine 5 may be implemented by a rules engine and a Bayesian network (inference) engine.

In one embodiment, the ACDM engine 20 may generate an indication of the confidence level of the automated clinical decision that it generated. If the confidence level exceeds a predetermined threshold level, the ACDM engine 20 may output the decision. If the confidence level is below the predetermined threshold level, engine 5 may output an indication that a decision cannot be made at this time, given the inputs it has received.

The disambiguation engine 30 determines whether a qualified clinician should treat a patient or whether the patient should be treated according to the automated clinical decision output from ACDM engine 20. This determination may be made, for example, using a rule set and/or machine-learning algorithm based on information from a tracking module 40 and information from a clinician database 50.

The tracking module 40 monitors the current location of clinicians, treatment devices, and/or patients within and/or outside of a medical facility. In one embodiment, the tracking module 40 may perform tracking operations based on a map of the medical facility, e.g., hospital. The tracking may be automatically performed, for example, based on signals received from RFID tags worn by the clinicians and/or patients or placed on the treatment devices. Additionally, or alternatively, tracking may be performed, for example, based on facial recognition software applied to images captured by cameras arranged throughout the facility. The locations of the clinicians may be stored based on clinical IDs retrieved, for example, from the clinician database 50. The locations of patients may be stored, for example, based on patient IDs derived from the EMR database 2.

In one embodiment, the tracking module 40 may code and track the activity of clinicians based on any of the following types of information: staff location (e.g., proximity to patient bed, in break room, proximity of other staff, etc.), fixed cameras and computer vision plus accelerometers (e.g., analysis of physical activity), body-worn cameras and computer vision (e.g., analysis of environment), and microphones, speech-to-text, and analysis performed by natural language programming (NLP) based on conversations picked up by microphones. Clinician availability may be inferred from the location (proximity to patient), activity (working on patient, taking a break, etc.), and/or the assessed needs of other patients. Telemedicine staff may be assumed to be available when on duty. The tracking module 40 may be informed of brief periods of non-availability (e.g., taking a break) based on entries by clinicians into an appropriate user interface.

The tracking module 40 may also track the location and/or availability of medical equipment in the facility. In one embodiment, a device connected to a patient may be associated with that patient via a user interface. The location of all devices may be tracked (e.g., via RFID and/or camera), and their availability may be checked by i) not being in motion and ii) not being associated with a patient. Optionally, a medical device may automatically move to a patient via some robotic locomotion and navigation system.

In one embodiment, the clinical decision may be disambiguated based on whether an onsite or remote authorized clinician is available for making a time-critical clinical decision. This may include, for example, the absence of a response to an alert sent to authorized clinicians within a certain amount of time. If no authorized clinician is determined to be available to make a time-critical clinical decision, the disambiguation engine 30 may select the automated clinical decision from the ACDM engine 20 to be implemented in place of treatment by a clinician. If a qualified clinician is available and has a higher rank relative to the ACDM engine, then the disambiguation engine 30 may select the clinician for purpose of treating a patient condition.

In one embodiment, the disambiguation engine 30 may make this selection by i) identifying which clinicians are qualified to treat a patient condition (as alerted by an ACDM system controller, which may or may not include the assessment module 10), ii) assessing the location and availability of the qualified clinicians; iii) prioritizing the available qualified clinicians (if any) by proximity to the patient; iv) sending a notification to the most suitable clinician(s); v) awaiting his/her/their response(s); and vi) proceed with making and implementing the decision when no response is received within a predetermined period of time.

In at least one embodiment, a clinician may be considered to be qualified to treat a certain patient condition based on having certain authorizations, certifications, experience, training, skill level, etc. This information may be determined beforehand, for example, based on policies of the medical facility or other healthcare organization, collaboration among physicians or other professionals, and/or administrator. In one embodiment, the qualification information may be stored in the clinician database 50 for use by the disambiguation algorithm 30 in selecting between a clinician or automated clinical decision for treating a patient.

In one embodiment, when a qualified clinician is determined to be available to treat a patient condition, the disambiguation engine 30 may be programmed to select a default position, e.g., may default to the clinician. In other embodiments, disambiguation engine 30 may be programmed to select the automated clinical decision from the ACDM engine 20 to direct treatment of the patient, for example, because the automated decision would prove to be a better option for treatment than the available clinician(s). The conditions for making this selection may be determined based on the decision algorithm implementing the disambiguation engine 30. For example, the disambiguation engine 30 may take into consideration how suitable or experienced an available clinician may be, e.g., given the clinician's expertise, specialty, years on the job, etc. At the time of decision making, if both ACDM and clinician are available, suitability could be the deciding factor for purposes of determining whether the automated decision or clinician is selected to treat the patient.

When the disambiguation engine 30 has selected the automated clinical decision, information corresponding to the automated clinical decision may be implemented by a controller 70. Automated implementation of the clinical decision may be controlled by a computer-based algorithm, which, in turn, controls operation of one or more therapeutic devices 110 at the patient location. An alerting system 120 and/or an order system 130 may also be subject to control by controller 70. In one embodiment, implementation of the automated clinical decision by the controller may be performed without human intervention. In other cases, one or more technicians or other personnel may at least partially implement treatment according to the automated clinical decision, once alerted by the alerting system 120. If the administration of drugs is involved, the ordering system 130 may prescribe the drugs and an appropriate dosage, for example, based on the patient data stored in the EMR database 2 and/or an assessment performed by the assessment module 10.

In one embodiment, the controller 70 implementing the automated clinical decision generates commands, signals, and/or other information for performing an action to treat the patient based on the automated clinical decision selected by the disambiguation engine 30. The controller 70 may perform these operations based on information from a fourth database 80 and a fifth database 90.

The fourth database 80 stores knowledge indicative of one or more clinical actions that are to be performed in implementing each type of automated clinical decision. For example, the fourth database 80 may store information indicating allowance or restrictions on which clinical decisions the ACDM engine 20 is authorized to make, given the patient condition to be treated. This may be determined, for example, using a mapping (or lookup) table which links various predetermined automated clinical decisions from the ACDM engine 20 with authorized clinical actions. In one embodiment, the table may map roles of clinicians (e.g., triage nurses, respiratory therapists, etc.) to actions or classes of actions.

In one embodiment, the clinical actions database 50 may store information indicative of clinical actions in a way that allows a decision to be made as to whether the actions can be automatically performed, and if so how. This may be accomplished, for example, by storing associated codified knowledge of what therapeutic equipment is located in the hospital, where the equipment is located, and whether the equipment is available for use on a given patient. In one embodiment, the information stored in database 50 may include a parameterized mapping of possible decisions to possible actions. Each action may be specified as a specific, parameterized interaction with the hospital informatics system, and may range from changing the setting of a ventilator to administering a drug to calling a code Example parameters include patient ID, patient location, device ID, device location, drug ID, drug dose, etc.

The fifth database 90 stores information indicating the types of diagnostic or therapeutic devices in the medical facility and whether those devices are available. The devices to be used in implementing the automated clinical decision may be determined by the controller based on the clinical actions knowledge output from database 80 for the patient condition to be treated. In one embodiment, the process of activating and controlling the therapeutic devices may be completely automated.

When a required diagnostic or therapeutic device is not at the patient location, the controller 70 may send a signal or message to the device. Such a device may be, for example, a ventilator, urinal, medicine dispenser, etc. In case the device is equipped with autonomous movement capability, the device may automatically move to the patient location in response to the signal based on, for example, location information (e.g., map information, GPS navigation, or other location information) of the medical facility.

When the diagnostic or therapeutic device is located in the patient room, the controller 70 may generate signals for moving the equipment into position relative to the patient and/or activating the equipment. Such a device may be, for example, a brain scanner, a camera, ventilator, or another piece of equipment.

When the diagnostic or therapeutic device is attached to the patient, the controller 70 may generate signals for activating the equipment. Examples of a therapeutic device include an intravenous (IV) drip, a ventilator, an infusion pump, and a defibrillator. Examples of diagnostic devices include a patient monitor, an ultrasound machine, and a brain scanner, as well as other devices. Again, in at least one embodiment, the operation of all of these devices may be performed automatically without little or no human intervention, although in some cases the devices may have to be connected to the patient by a human. The signals sent by the controller 70 to the diagnostic or therapeutic devices may be transmitted by wire or wirelessly (using one or more short-range communication protocols) or via the Internet. Information indicative of the automated clinical decision, the actions taken, and the therapeutic devices used, and the personnel involved in treating the patient (if any) may be included in an electronic medical record stored in EMR database 2 for future consideration.

In addition to the foregoing features, when the disambiguation engine 30 determines that the automated decision made by ACDM engine 20 is selected to treat the patient, the system may include a mechanism for an authorized person to override the decision made by the disambiguation engine 30. The override function may be programmed, for example, into the controller 70 that controls implementation of the automated clinical decision. In one embodiment, the override may be performed by an authorized person entering an instruction on a user interface of a computer, medical device, or communication device. In one embodiment, the override function may be performed based on a signal generated from an emergency ‘stop’ button in the patient room.

Use of the override mechanism may be determined, for example, based on a policy of the medical facility. In one embodiment, the override mechanism may stop the controller 70 from performing a particular action, and this may be documented in the EMR database 2 along with information indicating what action was overridden and by whom. If a mechanical stop button is used, a camera by the button (plus recognition software) may identify the person responsible for the override. Moreover, recognition may also be determined to confirm that the person pushing the button has override authority, to prevent use by unauthorized persons (e.g., visitors).

In one embodiment, a mechanism may be provided to learn, enter, review, edit, manage, and/or deploy all of the codified knowledge mentioned above. The ACDM engine 20 may make clinical decisions based on large amounts of codified knowledge, including medical knowledge and knowledge about staffing, medical devices, hospital layout etc. In one embodiment, the ACDM engine 20 may generate an automated clinical decision based on hospital-specific knowledge. All of the knowledge stored in the ACDM system may be subject to change, which, for example, may change the way the ACDM engine 20 and/or the disambiguation engine 30 behaves.

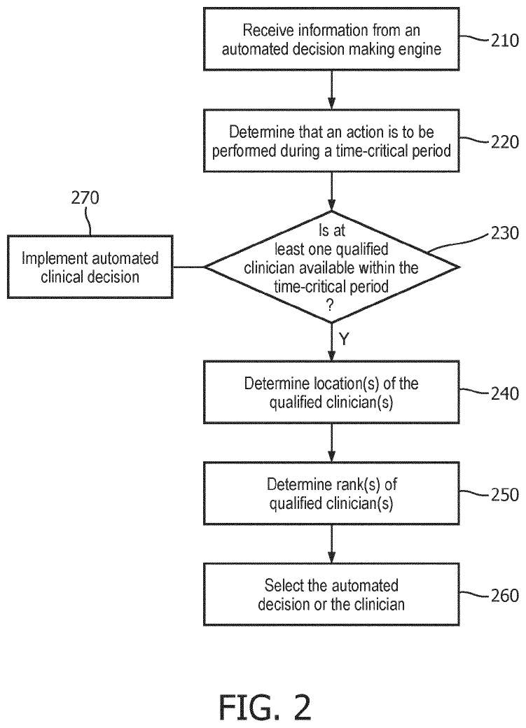

FIG. 2 illustrates an embodiment of a method for allocating healthcare resources, which, for example, may be performed by disambiguation engine 30. The method may be performed based on information and/or interaction with features of an automated clinical decision-making system, which, for example, may correspond to the system of FIG. 1 which includes ACDM engine 20. The method may be performed by a different type of automated decision-making system in another embodiment.

At 210, the method includes receiving information from automated clinical decision-making (ACDM) engine 20. The information is received by the disambiguation engine 30 and includes a clinical decision automatically generated by the ACDM engine for a patient condition. The system may be informed of the patient condition as a pre-condition to operation of the ACDM engine 20 and the disambiguation engine 30. The information received by the disambiguation engine 30 may or may not include an action that should be taken in order to implement the automated clinical decision. In one embodiment, the patient condition may be automatically determined, for example, based on monitoring equipment, cameras, or sensors, with or without human intervention.

In one case, the ACDM engine 20 may generate an automated clinical decision based on exigent circumstances. A variety of scenarios are possible. For example, one very useful application of the method involves directing care decisions for hospital patients facing life-threatening or time-critical conditions (or who otherwise require immediate treatment). Under these circumstances, the ACDM engine 20 may automatically generate a clinical decision when, for example, a monitored patient is determined to require acute attention. The ACDM engine may determine that an automated clinical decision is to be generated based on input from diagnostic medical equipment, monitors, authorized treatment schedules, and/or other information stored in one or more databases of the ACDM system as described herein.

At 220, the disambiguation engine may determine that an action is to be performed based on the clinical decision during a time-critical period. The urgency of time may be indicated in the information received from the ACDM engine 20 or may be evident from the automated clinical decision itself, as interpreted by the disambiguation engine 30 based on its attendant algorithm. In one embodiment, the ACDM engine 20 may determine the time-critical period based on information stored in one or more of the databases previously described. The ACDM engine 20 may use this information, along with other clinical decision-making knowledge stored in the database(s), to automatically generate the clinical decision that is sent to the disambiguation engine 30.

The time-critical period may vary for different patient conditions and/or treatment plans. For example, the time-critical period for a stroke victim or patient undergoing diabetic shock may be just a few minutes, whereas the time-critical period for a dialysis, sepsis, or post-operative patient may be a few hours. The information stored in the database(s) of the ACDM system may define these time periods and may be taken into consideration by the ACDM engine 20 for purposes of generating the clinical decision input into the disambiguation engine.

At 230, the disambiguation engine determines whether there is at least one qualified clinician available within the time-critical period to examine, treat, or perform another action regarding the patient. In making this determination, the disambiguation engine 30 may first determine which clinicians at the medical facility (e.g., hospital, doctor office, nursing home, rehabilitation center, home patient, etc.) are qualified to examine or treat the patient, given the patient condition and the automated clinical decision received from the ACDM engine 20. The clinician qualifications may be automatically determined by the disambiguation engine 30 based on a search of the clinician database. The information in the clinician database may indicate, for example, the areas of experience and/or expertise in a given area, the authorizations granted to the clinicians for performing examinations and administering treatment options, and/or the other information described herein. The information stored in the clinician database may be based on or derived from information stored in one or more other databases of the ACDM system.

At 240, the disambiguation engine 30 (or a controller coupled to the clinician database) may query the tracking module 40 to determine the real-time location of qualified clinicians. The tracking module 40 may determine the real-time location of clinicians passively (e.g., based on medical or administrative records) or actively (e.g., based on position monitoring or tracking devices associated with the clinicians). The tracking devices may be based on location data received from RFID tags, pagers, text messaging, facial recognition, camera imagery, or another type of sensor or software. The location information captured by these sensors may be uploaded to (or otherwise stored in) a database of the tracking module to provide a reliable indication of the location of clinicians throughout their shifts. This information may be input into the disambiguation engine 40 in order to determine the availability of qualified clinicians who can examine, treat, and/or administer other actions within the time-critical period for the patient.

In addition to clinician location and availability, the tracking module 40 may also store information indicative of patient location, which may be taken into consideration by the disambiguation engine 30 to determine qualified clinicians who may be in closest proximity to the patient and thus may be able to satisfy the requirements to be taken within the time-critical period.

At 250, once the availability of qualified clinicians has been determined, the disambiguation engine may determine the rank(s) of the clinician(s) relative to the clinical decision output from the ACDM engine 20. The rank(s) of the clinician(s) may be obtained, for example, by a query of the clinician database, which may store ranking information determined, for example, by the hospital administrator and clinical staff according to a hospital policy.

At 260, the disambiguation engine 30 generates a decision for treating or otherwise performing patient care within the time-critical period. The decision involves selecting either the automated clinical decision from the ACDM engine 20 or an available, qualified clinician. The selection may be made based on a decision algorithm that takes into consideration the availability, location, rank, authorizations, qualifications, and/or other information described herein relating to clinicians. If the disambiguation engine 30 selects the clinician, then the automated clinical decision output from the ACDM engine 20 is overridden in favor of the clinician. If the disambiguation engine 30 selects the automated clinical decision under circumstances where a qualified clinician is available within the time-critical period, then the algorithm controlling the disambiguation engine has determined that patient care may be better or more efficiently administered by implementing the automated clinical decision.

At 260, if the disambiguation engine 30 selects the automated clinical decision under circumstances where there is no qualified clinician available within the time-critical period, then the disambiguation engine 30 may serve as a back-up system that may save the life of the patient. The order of operations performed by the method may vary given the controlling algorithm and/or system or patient circumstances.

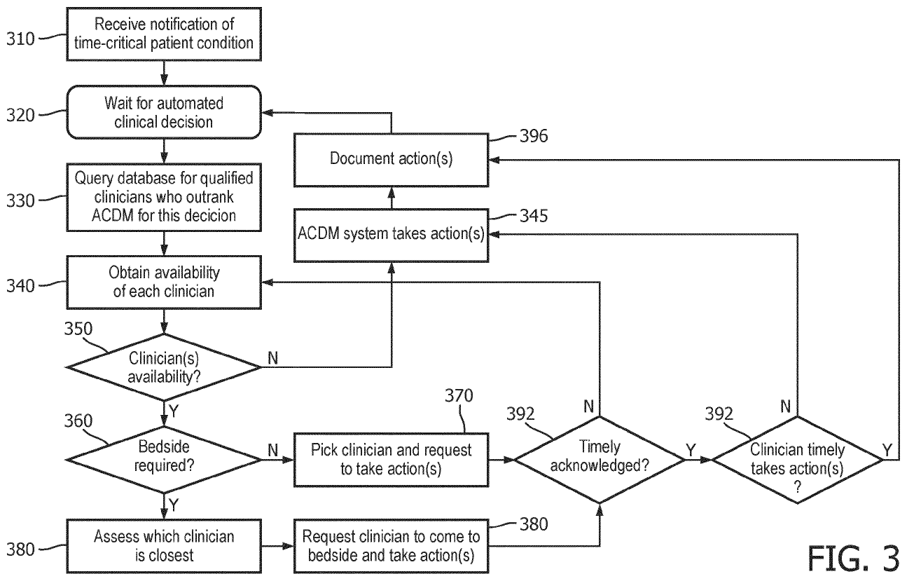

FIG. 3 illustrates another embodiment of a method for allocating healthcare resources, which, for example, may be performed by disambiguation engine 30. The method may be performed based on information and/or interaction with the ACDMS of FIGS. 1 and 2 and may be considered to be an example of a more specific implementation of the method of FIG. 2.

At 310, the method includes receiving notification of a patient condition that requires an action to be performed within a time-critical period. The notification may be received by the disambiguation engine 30, the ACDM engine 20, or another feature of the system of FIG. 1.

At 320, the disambiguation engine 30 waits for an automated clinical decision to be received from the ACDM engine 20. When the automated decision is received, the disambiguation engine 30 may not implement the decision right away, but rather may perform decision disambiguation according to its decision algorithm in order to provide the patient the best time-critical care possible given current circumstances in the medical facility. In one embodiment, the disambiguation engine 30 may perform one or more preliminary operations in preparation for implementing the automated clinical decision from the ACDM engine 20, should the automated decision be selected. This may occur, for example, when the automated clinical decision requires treatment or some other action that represents a substantial part of the time-critical period or which even exceeds that period. The disambiguation engine 30 may make this determination based on its control algorithm.

At 330, the disambiguation engine 30 queries one or more databases in the ACDM system to identify qualified clinicians who outrank the ACDM engine 20 relative to the patient condition under consideration.

At 340, the disambiguation engine 30 obtains from the tracking module 40 information indicative of the availability of the qualified technicians who were determined to outrank the ACDM engine 20 in operation 330. The availability is determined relative to qualified clinicians within the time-critical period.

At 350, the disambiguation engine 30 determines whether any clinicians are available based on the information obtained in operation 340. If no qualified clinicians are available within the time-critical period, then the disambiguation engine 30 may select the automated clinical decision from the ACDM engine 20 to be implemented, at 345. If at least one qualified clinician is available within the time-critical period, control algorithm of the disambiguation engine 30 proceeds to the next operation 360.

At operation 360, the disambiguation engine 30 queries one of the databases in the ACDM system (e.g., clinical decision making database, medical knowledge database, EMR database, or one of the knowledge databases) to determine whether the clinician is required to be present at the bedside of the patient in order to render a clinical decision and to take action.

At 370, if bedside presence is not required, the disambiguation engine 30 may select the clinician over the automated clinical decision output from the ACDM engine 20. When a plurality of clinicians are determined to be available in operation 350, the disambiguation engine 30 may select one of the clinicians randomly or based on a predetermined protocol or policy. A message or other form of alert may be sent to the selected clinician under control of the disambiguation engine, for example, along with patient status information, patient history, monitored data, recommended actions, and/or other information. In one embodiment, the automated clinical decision output from the ACDM engine 20 may even be sent to the selected clinician.

At 380, if bedside presence is required, the disambiguation engine 30 may perform an assessment to determine which qualified clinicians that outrank the ACDM engine 20 are closest in proximity to the patient. This determination may be made, for example, by querying the tracking module 40 which may store real-time information as to the location of the clinicians.

At 390, the disambiguation engine 30 sends a control signal to the alerting system to notify the selected clinician closest to the patient to go to the room of the patient to render a clinical decision and take associated action.

At 392, the disambiguation engine 30 awaits a response from the clinician selected and notified in operations 370 or 380. If the clinician does not respond within the time-critical period (or a predetermined period less than the time-critical period), control may return to operation 340 to ultimately allow the disambiguation engine 30 to select another available, outranking, qualified clinician, if possible. In one embodiment, the disambiguation engine 30 may implement the automated clinical decision from the ACDM engine 20, for example, based on the severity of the condition of the patient.

At 394, a determination is made as to whether the clinician actually showed up to treat the patient and/or whether the selected clinician was able to diagnose and/or treat (or perform some other action) for the patient in a timely manner, e.g., within the time-critical period. This determination may be made, for example, by tracking the location of the clinician. For example, if the clinician did not go to the patient room as instructed within the time period, the tracking module 40 may send a corresponding alert to the disambiguation engine 30. If the clinician went to the room of the patient but was unable to diagnose or treat the patient (or take any other required action), the disambiguation engine 30 may be alerted to this fact based on feedback from the clinician and/or diagnostic equipment and algorithms that is still indicating that the patient condition still persists.

If the clinician did not show up or perform an action in a timely manner (e.g., within the time-critical period), then the disambiguation engine 30 may direct implementation of the automated clinical decision from the ACDM engine 20. If the clinician did show up and perform an action in a timely manner, the clinical decision, diagnosis, action, and treatment (and all other operations performed for the patient episode) may be stored in electronic form in the EMR database, at 396.

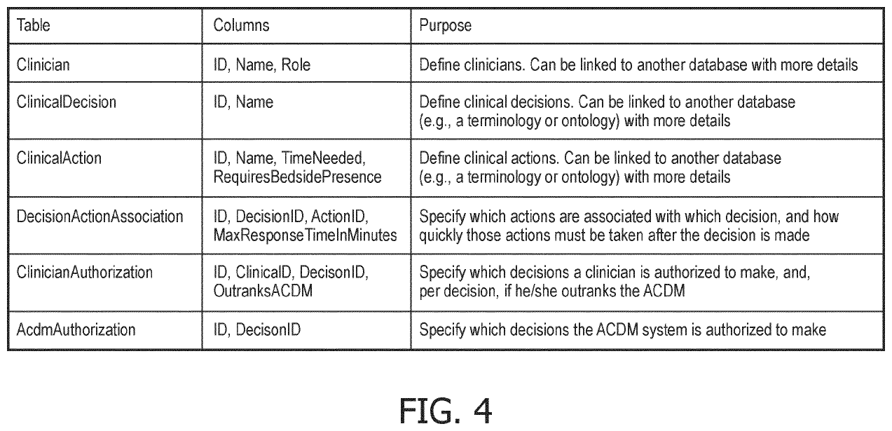

FIG. 4 illustrates an example of the contents of one or more of the databases (e.g., clinician database 50) in the system of FIG. 1. In one case, the contents shown in FIG. 4 may include information on a plurality of clinicians and their corresponding identification (IDs), names, and role (e.g., qualifications, experience, certifications, etc.).

The contents may also include information on a plurality of clinical decisions that may be generated, for example, by the ACDM engine 20. The clinical decisions may be identified, for example, by ID and name.

The contents may also include information on a plurality of clinical actions that may be identified by ID, name, time-requirements (e.g., including time-critical periods), requirements for bedside presence, and/or other information.

The contents may also include information on decision-action associations which may be identified by ID, DecisionID, ActionID, and maximum response time. This information may specify which actions are associated with corresponding decisions, and how quickly those actions must be performed after the decision is made.

The contents may also include information on clinical authorizations which may be identified by ID, ClinicianID, DecisionID, and ranking information. This information may specify which decisions a clinician is authorized to make and, per decision, whether the clinician outranks the ACDM engine for a give patient condition. The ranking may be determined, for example, based on a policy of a medical facility or a decision made by healthcare professionals.

The contents may also include information on ACDM Authorizations which may be identified by ID and DecisionID. This information may specify which decisions the ACDM engine is authorized to make. The contents of all of the tables/information in FIG. 4 may be linked with other databases.

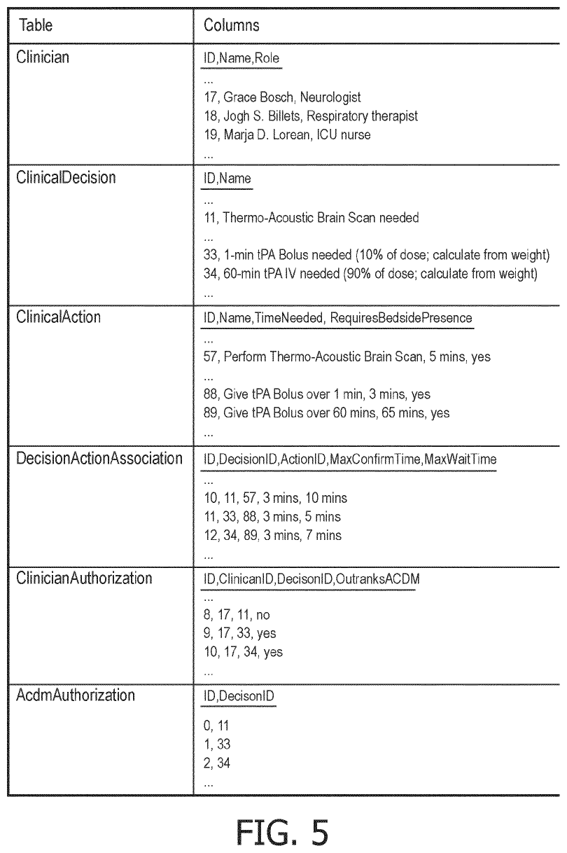

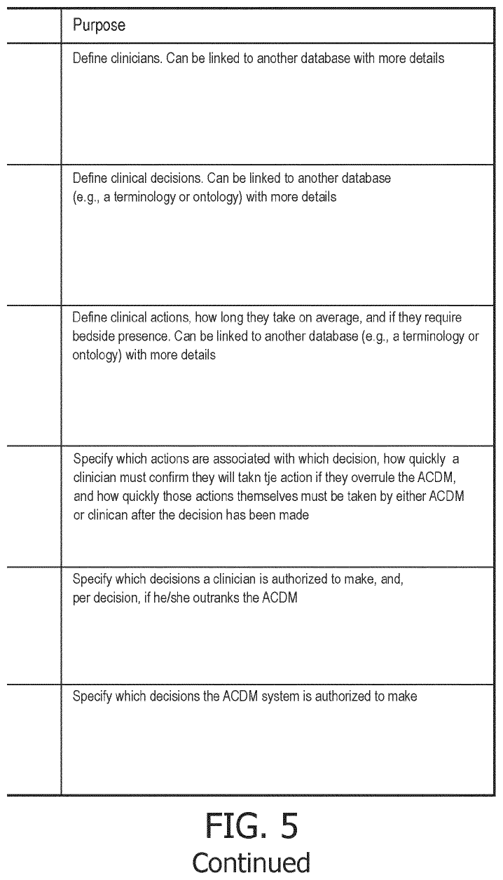

FIG. 5 illustrates an example of the contents illustrated in FIG. 4 filled out to include real-world information. The clinician information, for example, may define clinicians by name and medical expertise or position. The clinical decision information may specify various treatments, procedures, therapeutic devices, equipment, medications, and/or other information associated with remediating any one of a number of patient conditions. The clinical action information may define clinical actions relating to the clinical decisions. The decision-action association information may specify which actions are associated with which decisions, how quickly a clinician must confirm that he/she will take the action once notified and if they overrule the ACDM engine, and how quickly those actions themselves must be performed by a clinician or based on automated clinical decision output from the ACDM engine. The clinical authorization information specifies which decisions a clinician is authorized (or qualified) to make and, per decision, if the clinician outranks the ACDM engine for a given clinical decision or patient condition.

The ACDM authorization information specifies which decisions the ACDM engine is authorized to make.

The methods, processes, and/or operations described herein may be performed by code or instructions to be executed by a computer, processor, controller, or other signal processing device. The code or instructions may be stored in a non-transitory computer-readable medium in accordance with one or more embodiments. Because the algorithms that form the basis of the methods (or operations of the computer, processor, controller, or other signal processing device) are described in detail, the code or instructions for implementing the operations of the method embodiments may transform the computer, processor, controller, or other signal processing device into a special-purpose processor for performing the methods herein.

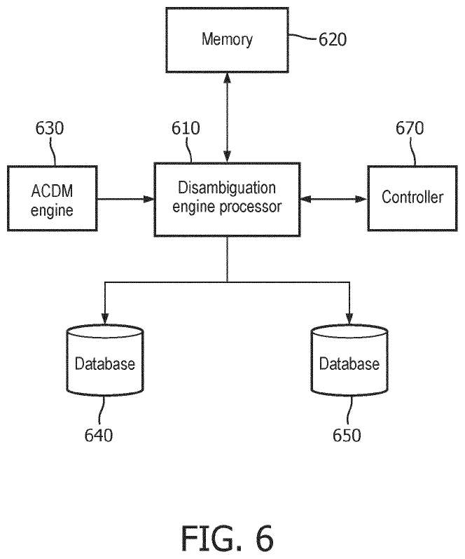

FIG. 6 illustrates an embodiment of a system which includes a computer-readable medium storing instructions for causing one or more processors to perform the operations of the system and method embodiments described herein. The system includes a processor 610 which executes instructions stored in a memory 620. The instructions may cause the processor 610 to perform the operations of the disambiguation engine 30. The processor also has a number of inputs to receive the automated clinical decision from the ACDM engine 630 and to input/output information, commands, queries, control signals, etc., to databases 640 and 650 and controller 670. The databases 640 and 650 may correspond, for example, to one or more of the databases discussed in the embodiment of FIG. 1. The controller 670 may correspond to controller 70 previously discussed.

### Use Case Example

The following example aims at further illustrating one or more embodiments of the system and method. In this example, it is assumed that the ACDM system takes part in the care of a patient by the name of Anne, an intubated ICU patient recovering from successfully treated pulmonary embolism. The ACDM system receives all data available from the EMR database 2, assessment module 10, and a variety of diagnostic and monitoring equipment, including but not limited to patient monitors, a ventilator, an infusion pump, and the output of a suite of algorithms that analyze the video feeds of several cameras aimed at Anne. In this example, the diagnostic and monitoring equipment may be connected to one or more of the modules or engines of the ACDM system, for example, through corresponding network connections or other communication links.

Suddenly, one of the cameras detects that Anne has developed Bell's palsy, which is a weakness in her facial muscles that makes the right half of her face droop. The assessment module 10, ACDM engine 20, or another processor of the ACDM system interprets the camera images and determines that Anne may be having a stroke, which is an acute, life-threatening disease caused by either a blood clot or a ruptured blood vessel in the brain. The assessment module 10 (or ACDM engine) may make this determination, for example, by running diagnostic deep learning inference on the camera images and/or other patient data against knowledge in one or more of the databases of the system. Analysis of ECG data automatically collected from monitoring sensors on Anne's body is processed by the ACDM engine. Based on this information, the ACDM engine 20 assesses that there is a 52% possibility of a cardiogenic stroke. As a result, the ACDM engine generates and implements an automated clinical decision to raise an alarm.

In this scenario, the intensive care unit (ICU) where the patient is located is equipped with a thermo-acoustic brain scanner that can diagnose stroke and differentiate between hemorrhagic stroke, ischemic stroke, and stroke mimic. The device is small and mounted on a robotic arm. Given the acute nature of Anne's condition, ACDM engine 20 automatically decides that a brain scan is required and implements the decision by issuing a command that controls the scanner to position itself relative to Anne's head. Within a minute, the ACDM engine 20 interprets the data from the scanner and assesses that Anne is suffering from an ischemic stroke (blood clot) with 92% certainty. Meanwhile, an ICU nurse has arrived and concurs with the assessment made by the ACDM of Anne's condition.

Based on the assessment of a 92% chance of ischemic stroke, the ACDM engine 20 generates an automated decision about treating Anne's condition. The ACDM engine 20 makes this determination based on the clinical decision-making knowledge stored in database 4. This knowledge includes a stroke protocol of the hospital, which indicates that Anne is to be given a 5 mg tPA bolus asap (based on her weight of 122 lbs.), followed by a 45 mg infusion, given by IV over 60 minutes. The ICU room is equipped with an Automated IV Medication Dispensing System that includes many of the most common and most urgent medications, including tPA.

At this point, the ACDM engine outputs the updated automated clinical decision and the action (e.g., administer medication) to be performed to the disambiguation engine 14. Even though the diagnosis and treatment (e.g., clinical decision and action, respectively) have been made with high confidence, and even though the ACDM engine could implement the decision and action, control is passed to the disambiguation engine 30.

Receipt of the clinical decision from the ACDM engine triggers the disambiguation engine 30 to identify at least one clinician available within the time-critical period (e.g., 5 minutes), determine the rank of the at least one clinician relative to the ACDM engine, and then select the at least one clinician or the clinical decision from the ACDM engine based on the rank within the time-critical period. For example, the disambiguation engine 30 may perform a query of the clinician database to determine that Dr. Grace Bosch, a neurologist, outranks the ACDM engine for the type of diagnostic and therapeutic decisions that have automatically be determined by the ACDM engine for Anne. The disambiguation engine 30 performs a query of the tracking module to determine that Dr. Bosch is onsite. At this point, the disambiguation engine 30 selects Dr. Bosch (clinician) for making the clinical decision and action relative to Anne, instead of implementing the clinical decision output from the ACDM engine.

In order to do so, the disambiguation engine 30 sends Dr. Bosch a message or alert to come to the Anne's room as soon as possible. The local protocol (e.g., information stored in one of the system databases) prescribes that the clinician should confirm within three minutes and be by the patient's bedside within five minutes after receiving this particular type of alert. In the example, the disambiguation engine 30 does not receive a response from Dr. Bosch after three minutes and proceeds with implementing the automated clinical decision from the ACDM engine 20, even though Dr. Bosch is available and outranks the ACDM engine 20 for this type of patient condition. For this purpose, the disambiguation engine 30 sends a control signal to the controller 70 to implement the automated clinical decision. The controller 70 controls the Automated IV Medication Dispensing System (one of the therapeutic devices) to automatically administer the tPA bolus to Anne within the time-critical period.

In this example, the disambiguation engine 30 serves as a vital back-up system that saves Anne's life when an outranking clinician is unavailable to respond within a time-critical period. For example, in this example, Dr. Bosch did not respond within a predetermined period (e.g., three-minute time limit) because she was delivering urgent care to another patient and was not able to respond to the alert that was sent to her until 10 minutes later, which would have increased Anne's degree of brain damage caused by the stroke and even threatened her life. Because an ischemic clot causes, on average, some 1.9 million neurons to die every minute, and because Dr. Bosch would not have been able to attend to Anne for at least 7 additional minutes (plus 2 more minutes to get to Anne's room), the disambiguation engine 30 may have saved an estimated minimum of (7+2)*1.9 ˜ 17 million neurons and many more synapses.

After the automated clinical decision from the ACDM engine 20 is implemented, all diagnostics, decisions, and actions taken may be recorded in the EMR database 2 to update Anne's health status for future assessments. Dr. Bosch may review the records concerning the stroke episode Anne experienced and agree or disagree as to how the disambiguation engine (and ACDM engine) performed. The decision algorithms used to implement these engines could then be updated with the feedback from Dr. Bosch, for purposes of rendering future decisions and disambiguation operations.

In accordance with one or more embodiments, a clinical decision may include one or more of the following: i) deciding whether or not a patent condition exists and/or whether or not an action should be taken to remediate or address that condition, ii) specifying the type and nature of the action, iii) if and when possible, actually implementing that action, and/or iv) documenting the decision, the reasoning behind the decision, and the action that was taken, if any. In performing these operations, the one or more modules or engines of the system may analyze all available patient data in combination including his/her current health status. The embodiments described herein may also provide more effective and immediate care to patients, especially when qualified clinicians are not available within a time-critical period. At the same time, one or more embodiments may reduce the cost of clinical care.

The modules, engines, processors, controllers, managers, and other information processing, calculating, and computing features of the embodiments herein may be implemented in logic which, for example, may include hardware, software, or both. When implemented at least partially in hardware, the modules, engines, processors, controllers, managers, and other information processing, calculating, and computing features may be, for example, any one of a variety of integrated circuits including but not limited to an application-specific integrated circuit, a field-programmable gate array, a combination of logic gates, a system-on-chip, a microprocessor, or another type of processing or control circuit.

When implemented in at least partially in software, the modules, engines, processors, controllers, managers, and other information processing, calculating, and computing features may include, for example, a memory or other storage device for storing code or instructions to be executed, for example, by a computer, processor, microprocessor, controller, or other signal processing device. Because the algorithms that form the basis of the methods (or operations of the computer, processor, microprocessor, controller, or other signal processing device) are described in detail, the code or instructions for implementing the operations of the method embodiments may transform the computer, processor, controller, or other signal processing device into a special-purpose processor for performing the methods described herein.

Although the various exemplary embodiments have been described in detail with particular reference to certain exemplary aspects thereof, it should be understood that the invention is capable of other example embodiments and its details are capable of modifications in various obvious respects. As is readily apparent to those skilled in the art, variations and modifications can be affected while remaining within the spirit and scope of the invention. Accordingly, the foregoing disclosure, description, and figures are for illustrative purposes only and do not in any way limit the invention, which is defined only by the claims.

1. A method for allocating healthcare resources, comprising:
(a) receiving information from an automated clinical decision-making (ACDM) engine, the information including an automated clinical decision for a patient condition;
(b) determining that an action based on the automated clinical decision is to be performed during a time-critical period;
(c) identifying at least one clinician available within the time-critical period;
(d) determining a rank of the at least one clinician relative to the automated clinical decision from the ACDM engine for the patient condition; and
(e) selecting the decision to be made and an associated action to be performed by the at least one clinician or based on the automated clinical decision, wherein (e) is performed based on the rank within the time-critical period and (b), (c), (d), and (e) are automatically performed using at least one machine-implemented algorithm.

2. The method of claim 1, wherein (b), (c), (d), and (e) are automatically performed using at least one machine-implemented algorithm without human intervention.

3. The method of claim 1, wherein (c) includes querying at least one database to identify the at least one clinician available within the time-critical period.

4. The method of claim 3, wherein (c) includes determining location and availability of the at least one clinician based on information received from a tracking module.

5. The method of claim 1, wherein (d) includes querying a database to determine qualifications of the at least one clinician based on the action to be taken for the patent condition.

6. The method of claim 1, wherein (e) includes selecting the action to be performed within the time-critical period based on the automated clinical decision when:
the automated clinical decision outranks the at least one clinician, or
no qualified clinician is available within the time-critical period.

7. The method of claim 6, further comprising generating one or more control signals to automatically perform the action.

8. The method of claim 7, wherein the one or more control signals activate a diagnostic and/or a therapeutic device to automatically perform the action.

9. The method of claim 1, wherein (e) includes selecting the action to be performed by the at least one clinician within the time-critical period when the at least one clinician is available within the time-critical period and outranks the automated clinical decision for the patient condition.

10. The method of claim 9, further comprising:
receiving information indicating that a condition has been satisfied; and
selecting the action to be performed based on the automated clinical decision.

11. The method of claim 10, wherein the condition includes one of:
the at least one clinician did not acknowledge a notification to perform the action, or
the at least one clinician did not perform the action within the time-critical period.

12. A non-transitory computer-readable medium storing instructions for causing one or more processors to:
(a) receive information from an automated clinical decision-making (ACDM) engine, the information including an automated clinical decision to treat a patient condition;
(b) determine that an action based on the automated clinical decision is to be performed during a time-critical period;
(c) identify at least one clinician available within the time-critical period;
(d) determine a rank of the at least one clinician relative to the automated clinical decision from the ACDM engine for the patient condition; and
(e) select the action to be performed by the at least one clinician or based on the automated clinical decision, wherein (e) is performed based on the rank within the time-critical period and (b), (c), (d), and (e) are automatically performed using at least one machine-implemented algorithm.

13. The medium of claim 12, wherein (e) includes selecting the action to be performed within the time-critical period based on the automated clinical decision when:
the automated clinical decision outranks the at least one clinician, or
no qualified clinician is available within the time-critical period.

14. The medium of claim 13, wherein the instructions cause the one or more processors to generate one or more control signals to automatically perform the action.

15. The medium of claim 14, wherein the one or more control signals activate a therapeutic device to automatically perform the action.

16. The medium of claim 12, wherein (e) includes selecting the action to be performed by the at least one clinician within the time-critical period when the at least one clinician is available within the time-critical period and outranks the automated clinical decision for the patient condition.

17. The medium of claim 16, wherein the instructions cause the one or more processors to receive information indicating that a condition has been satisfied and select the action to be performed based on the automated clinical decision.

18. The medium of claim 17, wherein the condition includes one of:
the at least one clinician did not acknowledge a notification to perform the action, or
the at least one clinician did not perform the action within the time-critical period.
